(19) United States
(12) Patent Application Publication
(10) Pub. No.: US 2011/0024319 A1
(43) Pub. Date: Feb. 3, 2011

(54) Product Name Indicia

(76) Inventors: Candace Dyan Krautkramer, Neenah, WI (US); Nicole Kay Leicht, Sherwood, WI (US); Linda Susan Huard, Appleton, WI (US); Christopher Patrick Worzalla, Hortonville, WI (US); Aida Elham Flick, Menasha, WI (US); Andrew Pottle Meurer, Neenah, WI (US)

Correspondence Address: KIMBERLY-CLARK WORLDWIDE, INC.;Tara Pohlkotte, 2300 Winchester Rd., NEENAH, WI, 54956, US

(21) Appl. No.: 12/533,458

(22) Filed: Jul. 31, 2009

(51) Int. Cl. A61F 13/84

(52) U.S. Cl. 206/438; 206/459.5

(57) ABSTRACT

A package includes a feminine-care article enclosed in a wrapper. The package includes product-name indicia, wherein the product-name indicia correspond to a deployment feature of the package, the feminine-care article, or the wrapper.

An array of packages includes a first package and a second package. The first package includes first product-name indicia and contains a first feminine-care article enclosed in a first wrapper. The first product-name indicia correspond to a first deployment feature of the first package, the first feminine-care article, or the first wrapper. The second package includes second product-name indicia and contains a second feminine-care article enclosed in a second wrapper. The second product-name indicia correspond to a second deployment feature of the second package, the second feminine-care article, or the second wrapper.

### BACKGROUND OF THE INVENTION

In the past, the marketing of feminine-hygiene products has often conveyed a narrative or story that associates menstruation with a medical or clinical condition. Even the phrase “feminine-hygiene product,” and words that describe these products and their use, such as “cycle,” or “protection,” are suggestive of a clinical condition. As a result, women may view menstruation and vaginal health as medical in nature, perhaps something that is not normal, rather than a natural part of who they are. This association between feminine-hygiene products and a medical condition may so shape a user's perception of these products that she may be reluctant to talk openly about her body, menstruation, and vaginal health. This is unfortunate because the more a woman understands herself, and what is going on with her body, the better.

Thus, if a manufacturer's choice of words, images, and color expressly or implicitly links these products to what is perceived as a clinical or medical narrative, then these same women may reject the manufacturer's products. As such, manufacturers have used product names that, by themselves, do not necessarily suggest a medical or clinical condition. For example, a number of companies have used product names that suggest the word “free”; possibly to suggest, at least some decades ago, freedom from supporting belts that were once used to hold pads in place: STAYFREE-brand feminine-care products; CAREFREE-brand feminine-care products; FREEDOM-brand feminine-care products; NEW FREEDOM-brand feminine-care products; and others. Manufacturers have also used product names that suggest jewelry (“PEARL-brand feminine-care products”); an ongoing presence or availability (“ALWAYS-brand feminine-care products”; “WHENEVER-brand feminine-care products”); comfort (“GENTLE GLIDE-brand feminine-care products”); an unquantifiable number (“INFINITY-brand feminine-care products”); and athletics (“SPORT-brand feminine-care products”).

However, none of these names correspond to the bold, non-traditional product-name indicia needed to more completely break with the medical/clinical marketing narratives of the past. Additionally, none of these names are reinforced every time a user undertakes an action corresponding to the product-name indicia.

### SUMMARY OF THE INVENTION

The present invention provides packages having bold, non-traditional product-name indicia; indicia unlike that used in the feminine-care category and indicia that we believe achieves the following: (1) people are more likely to remember the product; and (2) women are more likely to view menstruation, and products for menstruation, as free from the taboos, and free from the clinical/medical narrative, of the past. Specifically, we believe that product-name indicia corresponding to a deployment feature of the package, the feminine care article enclosed therein, and/or the wrapper containing the feminine care article are unexpected and fun and thus help to break with past marketing narratives. Also, the product-name indicia are reinforced in the mind of a user every time the user manipulates the deployment feature suggested by the product-name indicia.

As such, one aspect of the present invention provides a package containing a feminine-care article enclosed in a wrapper. The package includes a product-name indicia. The product-name indicia corresponds to a deployment feature of the package, the feminine-care article, or the wrapper.

In various embodiments of this aspect, the deployment feature may be an opening feature of the wrapper. In some embodiments, the opening feature may include a line of weakness, a frangible bond, or a strand.

In various embodiments of this aspect, the deployment feature may be an opening feature of the package. In some embodiments, the opening feature may include threads, a hinge, or a zipper.

In another aspect, the present invention provides a package containing a feminine-care article enclosed in a wrapper. The package includes a product-name indicia that corresponds to a deployment feature of the feminine-care article and the deployment feature is an activation feature of the feminine-care article.

In various embodiments of this aspect, the activation feature may be a mechanical stop. In some embodiments, the activation feature may be a peel strip on the feminine-care article.

In some embodiments, the deployment feature is a feature for manipulating the feminine-care article. In some embodiments, the feature for manipulating the feminine-care product is a grasping portion of an applicator.

In another aspect, the present invention provides an array of packages. The array includes a first package and a second package. The first package contains a first feminine-care article enclosed in a first wrapper. The first package includes a first product-name indicia, wherein the first product-name indicia corresponds to a first deployment feature of the first package, the first feminine-care article, or the first wrapper. The second package contains a second feminine-care article enclosed in a second wrapper. The second package includes a second product-name indicia, wherein the second product-name indicia corresponds to a second deployment feature of the second package, the second feminine-care article, or the second wrapper.

In various embodiments, the first product-name indicia are different from the second product-name indicia. In some embodiments, the first deployment feature is different from the second deployment feature.

In some embodiments, the first deployment feature is an activation feature of the first feminine-care article and the second deployment feature is a feature for manipulating the second feminine-care article.

In some embodiments, the first deployment feature is an activation feature of the first feminine-care article and includes a mechanical stop and the second deployment feature is a feature for manipulating the second feminine-care article and includes a grasping portion of an applicator.

### BRIEF DESCRIPTION OF THE DRAWINGS

FIG. 1 representatively illustrates a perspective view of a non-applicator tampon in a wrapper.

FIG. 2 representatively illustrates a perspective view of a package containing the non-applicator tampons of FIG. 1.

FIG. 3 representatively illustrates another exemplary non-applicator tampon in a wrapper.

FIG. 4 representatively illustrates a perspective view of a package containing the non-applicator tampons of FIG. 3.

FIG. 5 representatively illustrates a side view of an exemplary applicator tampon.

FIG. 6 representatively illustrates a perspective view of a package containing the applicator tampons of FIG. 5.

FIG. 7 representatively illustrates a feminine-care pad.

FIG. 8 representatively illustrates a perspective view of a package containing the feminine-care pads of FIG. 7.

FIG. 9 representatively illustrates a perspective view of an exemplary package in a closed position.

FIG. 10 representatively illustrates a perspective view of the package of FIG. 9 in an open position.

FIG. 11 representatively illustrates an exemplary array of packages of the present invention.

### DETAILED DESCRIPTION OF THE DRAWINGS

As discussed above, the present invention provides a package containing one or more feminine-care articles. The feminine-care articles are frequently enclosed in individual wrappers. The package includes product-name indicia that correspond to a deployment feature of the package and/or the feminine-care article and/or the wrapper.

The packages are frequently made of a plastic film, paperboard, nonwoven material, or similar substrates, or combinations thereof. In some embodiments the package substrates may include laminates or layered materials. Disposed on any of these substrates is a product-name indicia that includes a word corresponding to a deployment feature of the package and/or the wrapper and/or the feminine-care article. The indicia may be disposed on the package using any suitable means. Typically the indicia are disposed on a substrate by printing (e.g., ink-jet, or other forms of printing in which the ink is delivered without a roll or other device impacting or contacting the substrate; gravure, or other forms of printing in which a roll or other device contacts the substrate mechanically). Printing may be done as part of a series of unit-operations employed to make the packages (i.e., a printing operation that is “in-line” or part of the sequence of unit operations used to make the package). Alternatively, a substrate used to make the package may be printed first. Then the printed substrate may be used as a material input to the sequence of operations used to make the package (i.e., a printing operation that is “off-line” from the process used to make the package). In various embodiments other sequences of unit operations, whether in-line or off-line, may be used, so long as the finished package includes a substrate on which is disposed a product-name indicia, the combination having the inventive features disclosed herein.

The arrays and packages of the present invention may be made using any process and/or unit operation known to persons of skill for making, converting, and otherwise handling the material inputs (e.g., film and/or paperboard substrates for making packages or wrappers) and packages used to contain feminine-care products.

As used herein, the term “indicia” refers to distinctive marks, text, logos, colors, textures, illustrations, graphics, and the like. The product-name indicia include words that correspond to a deployment feature of the product and/or the wrapper and/or the package that a user manipulates during the normal course of using the product. For example, manipulation occurs when opening the wrapper, opening the package, activating an applicator, or similar actions. Exemplary product-name indicia that suggest deployment features include words like “turn”, “hold”, “click”, “snap”, “zip”, “grip”, “twist”, “strip”, “extend”, “peel”, and the like, and combinations thereof. These product-name indicia are believed to break with past marketing narratives and help reinforce the product-name indicia in the mind of the user during manipulation of the product deployment feature corresponding therewith.

As used herein, the term “correspond”, and derivatives thereof, means to suggest a function or structure of the deployment feature. As used herein, the term “deployment feature” refers to those features associated with opening a wrapper containing a product; or opening a package containing a plurality of individually wrapped products; or activating or otherwise manipulating a product before the product is used. The deployment feature may be an opening feature of the wrapper, such as a perforation, score line, or other line of weakness at which the wrapper is opened; a tab, or plurality of tabs, that a person can grasp, facilitating the pulling apart of the wrapper at a line of weakness, or the pulling apart of the wrapper at an area where the wrapper material is joined to itself (e.g., at a flange or seam); a string, plastic strip, or other component that a user pulls to open the wrapper; a tape tab that is used to attach the wrapper material to itself (e.g., a tape tab on a pouch that contains the feminine-hygiene product); and other such opening features known to persons in the art. Alternatively, the deployment feature may be an opening feature of the package, such as a hinged lid; a string, strip, zipper, or other component that a user manipulates to open the package. Alternatively, the deployment feature may correspond to an activation step that is typically carried out before the product is used. For example, some compact applicator tampons must first be extended before the absorbent pledget within can be ejected from the applicator and into the user's vaginal canal. The step of extending the applicator to its full length is an example of an activation step. Likewise, some feminine pads have peel strips that must first be removed to expose garment attachment adhesive before positioning the pad into the user's undergarments. The step of removing the peel strip is an example of an activation step.

In various embodiments, the textual portion of the product-name indicia may be of any font, size, or color amenable to the package dimensions, and associated substrate, on which the indicia is disposed. Furthermore, the textual portion of the product-name indicia may include non-traditional spellings, hyphens, and words from languages other than English. Product name indicia may also include graphics, symbols, icons, and the like. For example, if the product-name indicia include the term “turn”, then this term could be associated with the image of curvilinear arrows, oriented in opposing directions, to suggest or illustrate the textual component. Or, in another exemplary embodiment, if the product-name indicia include the term “click”, then this term could be bracketed, at its ends, by a plurality of outwardly directed lines, to suggest or illustrate the textual component, “click.”

In various embodiments, the product-name indicia may appear isolated from other text, or may be placed near the brand or sub-brand (or, alternatively, primary brand or secondary brand) with which the product-name indicia is associated. In some embodiments, the product-name indicia may itself be a brand.

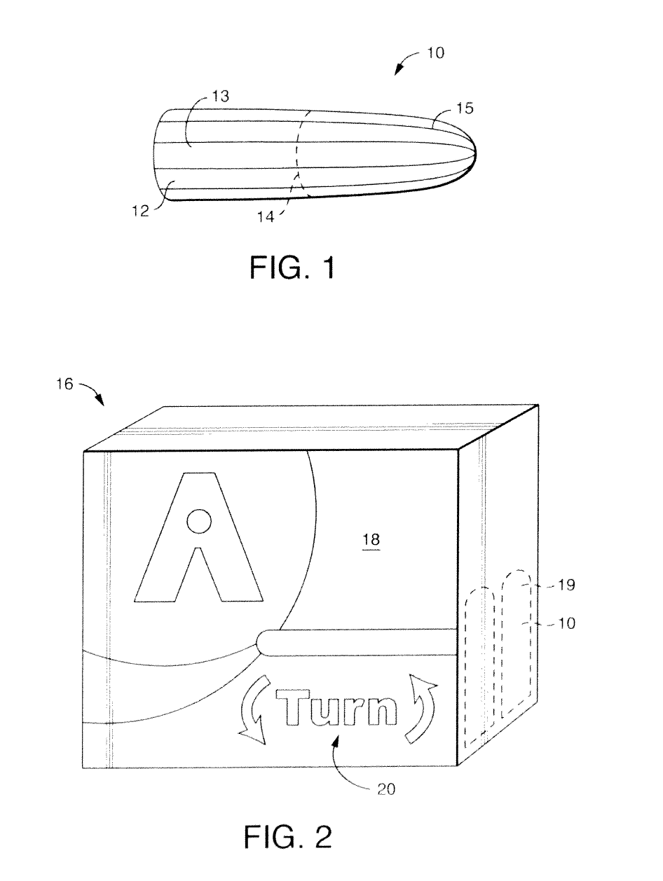

In an exemplary embodiment, the feminine-care product may be a non-applicator tampon 10 as illustrated in FIG. 1. The bullet-shaped tampon 10 can be wrapped with a film wrapper 12 such that the film wrapper 12 assumes the same basic shape as the enclosed tampon 10. This film wrapper 12 can include a circular perforation 14 around the circumference of the wrapper somewhere about the middle of the tampon. A user may open the film wrapper 12 by using the thumb and forefinger of one hand to grasp one portion 13 of the film wrapper 12 above this circular perforation 14 and the thumb and forefinger of the other hand to grasp the opposing portion 15 of the film wrapper 12 below the perforation 14. By rotating these portions in opposite directions, the perforation 14 gives way, giving the user access to the non-applicator tampon 10 within. A product-name indicia that corresponds to this opening feature of the wrapper, such as “turn”, or the like, may then be used on the substrate of the package containing the wrapped non-applicator tampons, the wrapper around each non-applicator tampon, or both the packaging and the wrapper.

For example, referring now to FIG. 2, a package 16 is illustrated with substrate 18. The package 16 can include any suitable product 19 therein, such as one or more of the non-applicator tampons 10 of FIG. 1. The substrate 18 of the package 16 includes product-name indicia 20 which correspond to the opening feature (circular perforation 14) of the tampon 10. The exemplary product-name indicia 20 “turn” is non-traditional and does not reinforce the undesirable associations between the tampon 10 and clinical or medical treatments. Furthermore, the product-name indicia 20 are unexpected and fun, in that they suggest an action undertaken by a user to open the product. Additionally, a user is more likely to associate the product-name indicia 20 “turn” with the turning action undertaken with each use of the product 10. This association reinforces the connection between the product-name indicia and the deployment feature in the mind of the consumer.

In a similar embodiment, the feminine-care product may be a tampon dispensed from an applicator. The applicator tampon may be wrapped with a film having a circular perforation around the circumference of the wrapper. A user may open the wrapper by using the thumb and forefinger of one hand to grasp one portion of the film above this circular perforation and the thumb and forefinger of the other hand to grasp the opposing portion of the wrapper below the perforation. By rotating these portions in opposite directions, the perforation gives way, giving the user access to the applicator tampon within. A product-name indicia that corresponds to this opening feature of the wrapper, such as “turn”, or the like, may then be used on the substrate of the packaging containing the wrapped applicator tampons, the wrapper around each applicator tampon, or both the packaging and the wrapper.

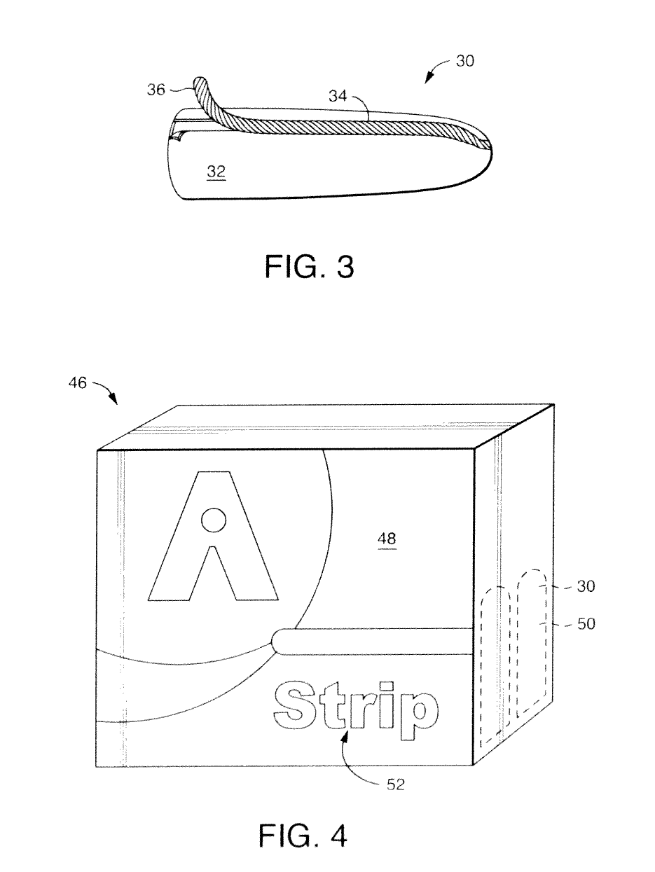

Referring now to FIG. 3, another exemplary feminine-care product is illustrated generally at 30. The feminine-care product in this example is a non-applicator tampon wrapped in a film 32. This film wrapper 32 can include a removable strip 34 of material such as one or more threads or strands. A user may open the film wrapper 32 by grasping one end 36 of the removable strip 34 of material and pulling the strip 34 to remove it from the film wrapper 32. In various embodiments the removal of the strip 34 severs or otherwise opens the film wrapper 32 and allows access to the feminine care product 30 housed therein. A product-name indicia that corresponds to this opening feature of the wrapper, such as “strip”, or the like, may then be used on the substrate of the packaging containing the wrapped feminine-care product, the wrapper around each feminine-care product, or both the package and the wrapper.

For example, referring now to FIG. 4, a package 46 is illustrated with substrate 48. The package 46 can include any suitable product 50 therein, such as one or more of the feminine care products 30 of FIG. 3. The substrate 48 of the package 46 includes product-name indicia 52 which correspond to the opening feature (removable strip 34) of the feminine care product 30. The exemplary product-name indicia 52 “strip” is non-traditional and does not reinforce the undesirable associations between the feminine care products 30 and clinical or medical treatments. The product-name indicia 52 suggest an action undertaken by a user to open the product. Thus, a user is more likely to associate the product-name indicia 52 “strip” with the act of removing the strip 34 each time the product 30 is used.

In some cases, the wrapper used to contain the product does not include a circumferential line of weakness. The line of weakness may extend for some length less than the entire circumference of a cylindrically wrapped product. And the line of weakness need not be straight, but can be curved, sinusoidal, zigzag-shaped, or have some other design. Furthermore, the line of weakness may be at different angles (i.e., the line of weakness need not be perpendicular or parallel to a side edge of the wrapper).

A wrapper can include a line of weakness that extends in a line along some or all of the length or width of the wrapper. A user opens the wrapper at this line of weakness, retrieving the product contained within the wrapper. Again, the present invention provides product-name indicia that correspond to this deployment feature of the product (in this case, an opening feature associated with the wrapper). If the line of weakness is along the entire length of the wrapper near one of the wrapper's longitudinal edges, and if a user opens the wrapper by grasping each of the opposing sides of the wrapper along this length and pulling in opposing directions, then product-name indicia that include terms such as “zip” and the like may be suggestive of the deployment feature.

Similar product-name indicia may be used if the line of weakness is in the transverse direction (that is, the direction perpendicular to the longer, longitudinal dimension of the product, unless of course the product is square-shaped, which may be the case for some pads in their folded state). In some cases the product-name indicia can correspond to the shape of the line of weakness, or the orientation of the line of weakness.

In another exemplary embodiment, the feminine-care product may be a tampon wrapped with a film wherein the film extends beyond one or both ends of the tampon. This film wrapper can be twisted to enclose the tampon similar to a wrapped piece of candy. A user may open the wrapper by using the thumb and forefinger of one hand to grasp the tampon and the thumb and forefinger of the other hand to untwist the extended portion of the wrapper. By untwisting the wrapper the user is able to access the tampon within. A product-name indicia that corresponds to this opening feature of the wrapper, such as “twist”, or the like, may then be used on the substrate of the packaging containing the wrapped tampon, the wrapper around each tampon, or both the package and the wrapper. The product-name indicia “twist” or the like suggest the relevant deployment feature and reinforces the name in the mind of the user.

Some feminine-care products include a plastic or paperboard applicator that contains an absorbent pledget or a pessary device. A woman inserts the applicator into her vaginal canal and, by depressing a plunger portion of the applicator, ejects the absorbent pledget or pessary device from the applicator into the vaginal canal. The applicator is then withdrawn and disposed of, leaving the pledget or pessary device in the vagina for its intended purpose. Some applicators are compact, and before they can be used to eject the absorbent pledget or pessary device contained within, they must be activated for use.

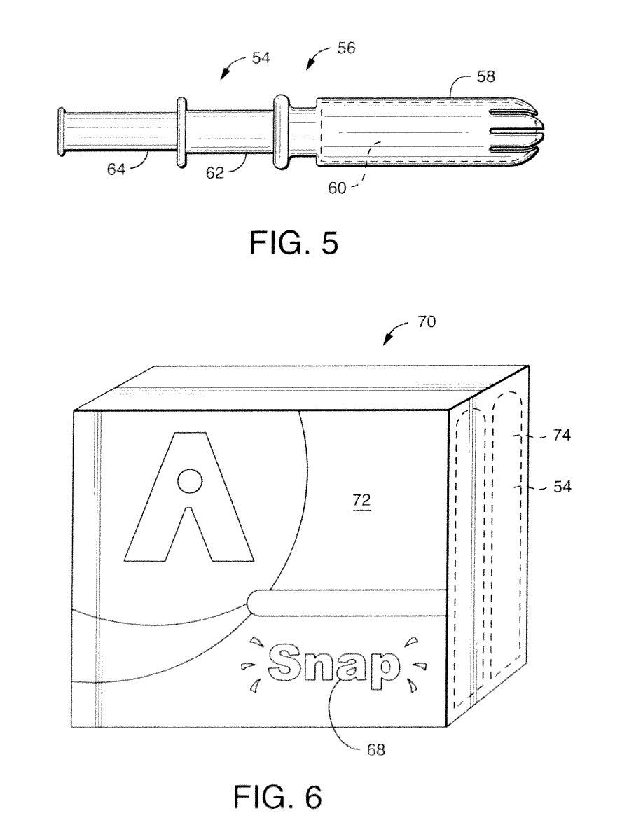

For example, as illustrated in FIG. 5, a feminine care product 54 includes an applicator 56 and a pledget 60. The applicator 56 includes three portions: a barrel portion 58 containing the pledget 60; a telescoping portion 62 at least partially inside the barrel portion 58, with the telescoping portion 62 capable of sliding within the barrel portion 58; and a plunger portion 64 at least partially inside the telescoping portion 62, with the plunger portion 64 capable of sliding within the telescoping portion 62. In other words, the applicator 56 is somewhat like a telescope made of two, three, or more cylindrical portions of decreasing diameter, one inside the other, such that the telescope can assume a compact length before use, and then be fully extended when used. So it is with some telescoping applicators. In the case of a telescoping applicator 56, the plunger portion 64 must be pulled so that it reaches a mechanical stop inside the telescoping portion 62 and then engages into place. In this way the plunger portion 64, when depressed, slidably moves both the plunger portion 64 and the telescoping portion 62, which are now interconnected by virtue of the user fully extending the applicator 56 and engaging the plunger portion 64 into place within the telescoping portion 62. Product-name indicia 68 that are suggestive of such a product can include terms like “extend”, “snap”, “click” and the like. The product-name indicia 68 may then be used on the substrate of the packaging containing the wrapped feminine-care product 54, the wrapper around each feminine-care product 54, or both the package and the wrapper.

For example, referring now to FIG. 6, a package 70 is illustrated with substrate 72. The package 70 can include any suitable product 74, such as one or more of the feminine care products 54 of FIG. 5. The substrate 72 of the package 70 includes product-name indicia 68 which correspond to the activation feature (snapping the applicator 56 into place) of the feminine care product 54. The exemplary product-name indicia 68 “snap” is non-traditional and does not reinforce the undesirable associations between the feminine care products 54 and clinical or medical treatments. The product-name indicia 68 suggest an action undertaken by a user to open the product. Thus, a user is more likely to associate the product-name indicia 68 “snap” with the act of snapping the applicator 56 into place each time the product 54 is used.

In a similar embodiment, some applicators may be designed such that activation of the applicator requires rotational movement of one or more elements of the applicator. For example, the applicator may initially be of some more compact length. A user then rotates some portion of the applicator, such as the plunger, which then extends the applicator to its full length prior to insertion of the applicator into the vagina. Packages containing such a product form can include product-name indicia that include terms like “turn”, and the like that are suggestive of this particular deployment feature.

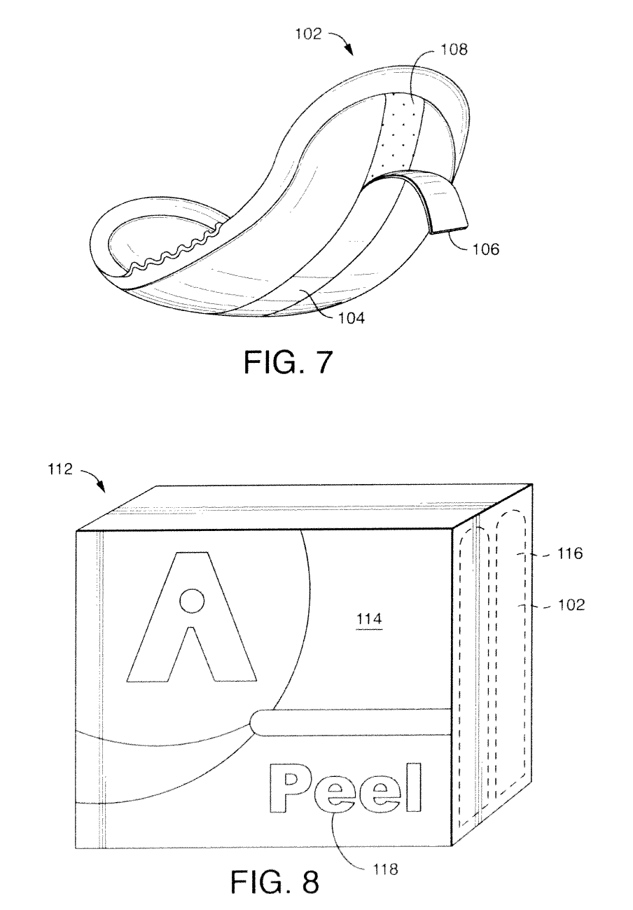

Many feminine pads have peel strips that must first be removed to expose garment attachment adhesive before positioning the pad into the user's undergarments. For example, referring now to FIG. 7, an exemplary feminine-care product is illustrated generally at 102. The feminine-care product in this example is an absorbent pad suitable for placement in a wearer's undergarment to absorb menses and other body exudates. The absorbent pad 102 can include a removable strip of material frequently referred to as a peel strip 104. A user may remove the peel strip 104 by grasping one end 106 and pulling to remove it from the absorbent pad 102. Removal of the peel strip 104 exposes garment attachment adhesive 108 that is suitable for adhering the absorbent pad 102 to a wearer's undergarments. A product-name indicia that corresponds to this deployment feature, such as “peel”, or the like, may then be used on the substrate of the packaging containing the feminine-care product, the wrapper around each feminine-care product, or both the package and the wrapper.

For example, referring now to FIG. 8, a package 112 is illustrated with substrate 114. The package 112 can include any suitable product 116, such as one or more of the absorbent pads 102 of FIG. 7. The substrate 114 of the package 112 includes product-name indicia 118 which correspond to the activation feature (peel strip 104) of the feminine care product 102. The exemplary product-name indicia 118 “peel” is non-traditional and does not reinforce the undesirable associations between the feminine care products 102 and clinical or medical treatments. The product-name indicia 118 suggest an action undertaken by a user to activate the product. Thus, a user is more likely to associate the product-name indicia 118 “peel” with the act of removing the peel strip 104 each time the product 102 is used.

Products may also include features that assist the user when manipulating the product. For example, some applicators for ejecting an absorbent pledget or pessary device may include materials or structure such that a person can more easily manipulate the applicator. Packages containing such a product form can include product-name indicia such as “hold”, “grip”, or the like that are suggestive of this particular deployment feature.

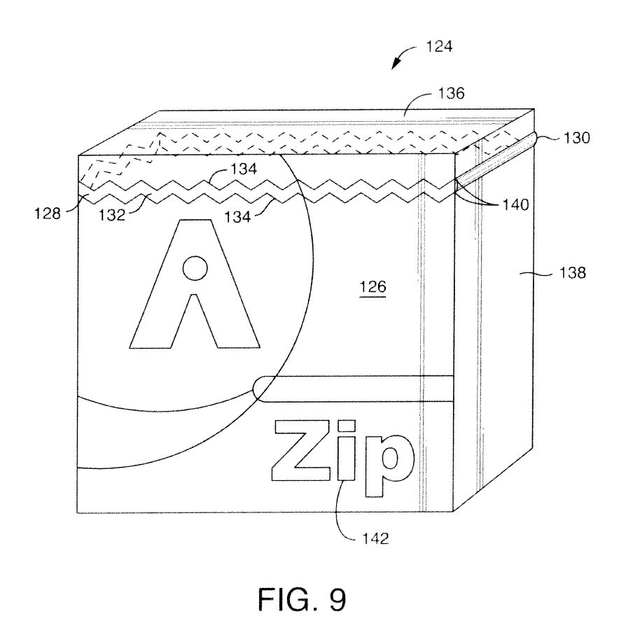

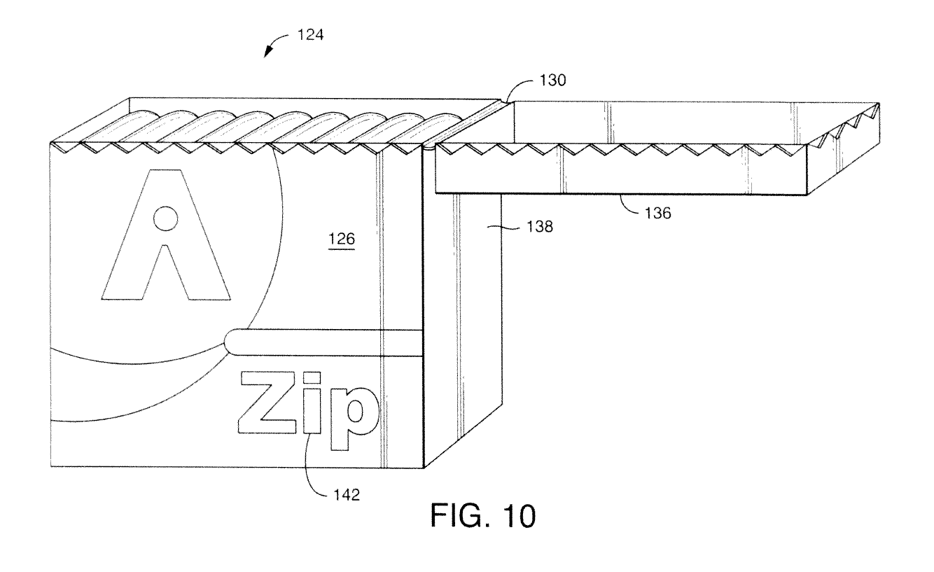

In some embodiments, the package may include an opening feature wherein the lid of the package is joined to the body of the package via one or more hinges. Packages containing such a feature can include product-name indicia such as “flip”, “hinge”, or the like that are suggestive of this particular deployment feature. Referring now to FIGS. 9 and 10, a package 124 is illustrated in a closed position and an open position respectively. The package 124 may include any suitable feminine care product therein. The package 124 includes a substrate 126, an opening feature 128, and a hinge 130. The opening feature 128 in this embodiment is a removable strip 132 that has a zigzag configuration. The removable strip 132 is defined by two lines of weakness 134. The removable strip 132 also divides the package 124 into a lid portion 136 and a body portion 138. To access the products within the body portion 138 of the package 124 a user grasps one end 140 of the removable strip 132 and pulls to break the lines of weakness 134 and remove the removable strip 132 from the package 124. Removal of the removable strip 132 partially releases the lid portion 136 from the body portion 138.

In the illustrated embodiment, the removable strip 132 is located on three sides of the package 124. The fourth side of the package includes the hinge 130. The hinge 130 may be made of any suitable material or construction adapted to pivotally secure the lid portion 136 to the body portion 138. The hinge 130 allows the lid portion 136 to be pivotally moved back and forth between the open position illustrated in FIG. 10 and the closed position illustrated in FIG. 9.

The substrate 126 of the package 124 includes product-name indicia 142 which suggests the activation feature (removing the strip 132) of the package 124. The exemplary product-name indicia 142 “zip” is non-traditional and does not reinforce the undesirable associations between the feminine care products contained in the package 124 and clinical or medical treatments. The product-name indicia 142 suggest an action undertaken by a user to open the package. Thus, a user is more likely to associate the product-name indicia 142 “zip” each time the package is opened. In similar embodiments, the opening feature 128 may be a traditional zipper (parallel rows of interconnecting teeth with a slider) suitable for releasing the lid portion 136 from the body portion 138. In yet other similar embodiments, the product-name indicia “hinge” would also be suitable as it is suggestive of an opening feature of the package 124.

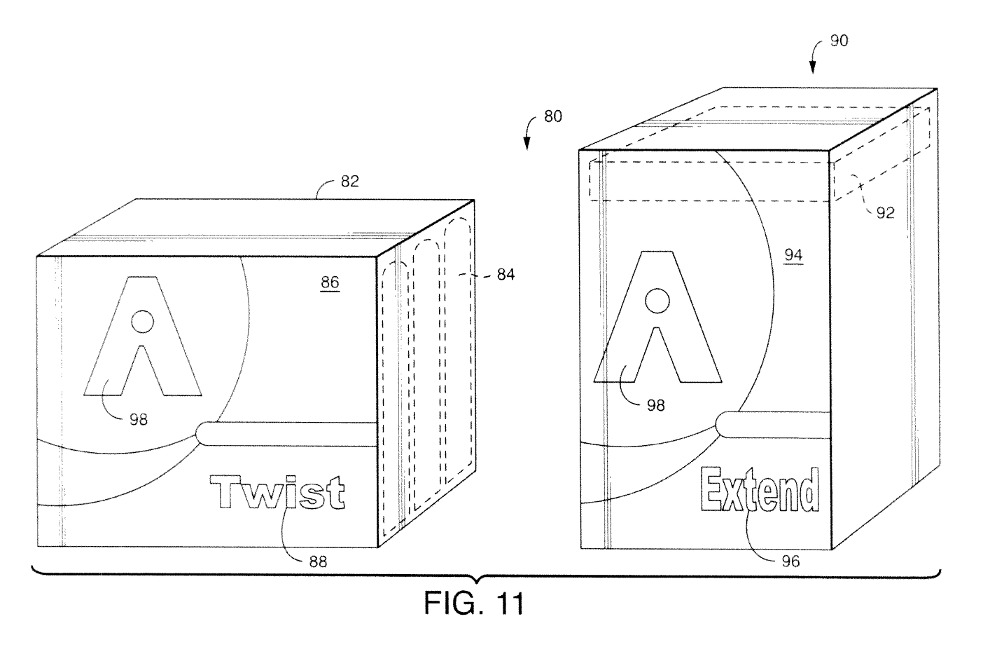

In various embodiments, any two or more of the embodiments described herein may form all or part of an array of packages containing feminine care articles. For example, referring now to FIG. 11, an array 80 includes a first package 82 and a second package 90. The first package 82 contains a first feminine-care product 84 individually wrapped in a first wrapper. The first package 82 also includes a first printed substrate 86 having a first product-name indicia 88 disposed thereon. The first product-name indicia 88 corresponds to a deployment feature of the first package 82, the first wrapper, and/or the first feminine-care product 84 as described herein. In the illustrated embodiment, the first product-name indicia 88 are “twist” which is suggestive of a deployment feature of the first feminine-care product 84 contained in the first package 82.

The array 80 also includes a second package 90 that contains a second feminine-care product 92 individually-wrapped in a second wrapper. The second package 90 may also include a second printed substrate 94 having a second product-name indicia 96 disposed thereon. The second product-name indicia 96 corresponds to a deployment feature of the second package 90, the second wrapper, and/or the second feminine-care product 92 as described herein. In the illustrated embodiment, the second product-name indicia 96 are “extend” which is suggestive of a deployment feature of the second feminine-care product 92 contained in the second package 90.

In various embodiments, the first product-name indicia 88 are different from the second product-name indicia 96 as illustrated in FIG. 11. However, in this exemplary array 80, both the first package 82 and the second package 90 have indicia of a brand 98 common to both (e.g., indicia of a master or primary brand). The common brand indicia 98 are disposed on both the first substrate 86 of which the first package 82 is composed and on the second substrate 94 of which the second package 90 is composed. Such indicia are not required.

In some embodiments, the first feminine-care article may be a telescoping applicator tampon contained within a first package including the product-name indicia “click”. The product-name indicia “click” corresponds with the deployment feature of the telescoping applicator tampons. Additionally, the second feminine-care article may be an applicator tampon having a distinctive grasping feature contained within the second package which includes the product-name indicia “grip”. The product-name indicia “grip” corresponds with the deployment feature of the applicator tampon.

As noted above, packages may be made from a variety of different substrates on which may be disposed product-name indicia, brand indicia, sub-brand indicia, and other text, symbols, icons, graphics, and the like. For example, a package may be made of paperboard and be in the form of a parallelepiped, oval, cone, or other shape having a volume in which individually wrapped items may be placed and contained before use.

Alternatively, the package may be made of a plastic film. Typically the film will conform, at least generally, to the contents within the package comprising the film. Often, packages that include plastic film take the basic shape of a parallelepiped.

Packages may comprise other indicia, navigational aids, and features. For example, the packages may comprise one or more transparent or translucent areas through which the contents of the package may be viewed. Such transparent or translucent areas may be of any size, shape, or location. In another example, the packages often include navigational aids; i.e., text or graphics that help a potential customer pick a product from among a plurality of products. For example, manufacturers may show simple silhouettes of an array of products, with each silhouette showing one of the available products in the array (or product line). Under each silhouette is text identifying that particular product (e.g., a series of 3 silhouettes, with each silhouette, in sequence, corresponding to “Regular,” “Long,” and “Overnight” feminine-care pad products; under each silhouette is one of the aforementioned words, and one caption above the silhouettes can be something like “Absorbency”).

While the invention has been described in detail with respect to specific embodiments thereof, it will be appreciated that those skilled in the art, upon attaining understanding of the foregoing will readily appreciate alterations to, variations of, and equivalents to these embodiments. Accordingly, the scope of the present invention should be assessed as that of the appended claims and any equivalents thereto. Additionally, all combinations and/or sub-combinations of the disclosed embodiments, ranges, examples, and alternatives are also contemplated.

1. A package containing a feminine-care article enclosed in a wrapper, the package comprising a product-name indicia, wherein the product-name indicia corresponds to a deployment feature of the package, the feminine-care article, or the wrapper.

2. The package of claim 1 wherein the deployment feature is an opening feature of the wrapper.

3. The package of claim 2 wherein the opening feature comprises a line of weakness.

4. (canceled)

5. The package of claim 2 wherein the opening feature comprises a strand.

6. The package of claim 1 wherein the deployment feature is an opening feature of the package.

7. The package of claim 6 wherein the opening feature comprises threads.

8. The package of claim 6 wherein the opening feature comprises a hinge.

9. The package of claim 6 wherein the opening feature comprises a zipper.

10. A package containing a feminine-care article enclosed in a wrapper, the package comprising a product-name indicia that corresponds to a deployment feature of the feminine-care article, wherein the deployment feature is an activation feature of the feminine-care article.

11. The package of claim 10 wherein the activation feature comprises a mechanical stop.

12. The package of claim 10 wherein the activation feature comprises a peel strip on the feminine-care article.

13. (canceled)

14. (canceled)

15. An array of packages, the array comprising a first package and a second package, wherein
the first package contains a first feminine-care article enclosed in a first wrapper, the first package comprising a first product-name indicia, wherein the first product-name indicia corresponds to a first deployment feature of the first package, the first feminine-care article, or the first wrapper and
the second package contains a second feminine-care article enclosed in a second wrapper, the second package comprising a second product-name indicia, wherein the second product-name indicia corresponds to a second deployment feature of the second package, the second feminine-care article, or the second wrapper.

16. The array of claim 15 wherein the first product-name indicia is different from the second product-name indicia.

17. The array of claim 16 wherein the first deployment feature is different from the second deployment feature.

18. (canceled)

19. The array of claim 17 wherein the first deployment feature is an activation feature of the first feminine-care article and comprises a mechanical stop and the second deployment feature is a grasping portion of an applicator.
